(19) United States
(12) Patent Application Publication
(10) Pub. No.: US 2022/0273176 A1
(43) Pub. Date: Sep. 1, 2022

(54) CLEANING MEDIUM SCANNER SYSTEM

(71) Applicant: Francis Schneider, Omaha, NE (US)

(72) Inventor: Francis Schneider, Omaha, NE (US)

(21) Appl. No.: 17/683,068

(22) Filed: Feb. 28, 2022

Related U.S. Application Data
(60) Provisional application No. 63/155,199, filed on Mar. 1, 2021.

(51) Int. Cl. A61B 5/00; G01N 21/88

(52) U.S. Cl. CPC A61B 5/0082; G01N 21/8806; G01N 2021/8887; G01N 33/4833

(57) ABSTRACT

A system and method for testing a cleaning medium, such as toilet paper, after wiping a perineum is disclosed. The system includes an optical sensor configured to scan the cleaning medium, a user interface, an output device, and a controller. The controller is configured to receive sensor input from the optical sensor, determine the presence of a biological substance, such as feces, on the cleaning medium based on the sensor input, generate a data signal based on the presence of the biological substance, and transmit the data signal to the output device. The system may determine if the cleaning medium is soiled or unsoiled, and may also detect the presence of blood and other types of biological material.

### CROSS-REFERENCE TO RELATED APPLICATIONS

This application claims the benefit and priority to U.S. Provisional Patent Application No. 63/155,199 filed Mar. 1, 2021, which is hereby incorporated by reference in its entirety.

### TECHNICAL FIELD

The present invention generally relates to personal hygiene, and in particular to a personal hygiene assurance device and method.

### BACKGROUND

Traditional methods for cleaning the perineum region of the body after defecation, urination and/or menstruation (e.g., wiping with tissue paper, commonly referred to as toilet paper) may be a challenge for a portion of the population. For example, a visually impaired person may not be able to detect whether the perineum is properly cleaned, as they may be unable to inspect whether the tissue paper they are using is soiled or unsoiled after wiping. In another example, a person that dislike viewing or is sickened by the sight of feces may be hesitant to look at soiled tissue paper. The inability to inspect soiled paper may also be a health risk to a person unknowingly suffering from a disease with symptoms that include blood on toiled paper after wiping, such as inflammatory bowel disease (IDS) or colorectal cancer.

Therefore, it would be advantageous to provide a system and method that overcomes the shortcomings described above.

### SUMMARY

A system is disclosed. In one or more embodiments, the system includes an optical sensor configured to scan a cleaning medium and transmit sensor input. In one or more embodiments, the system further includes an output device, a user interface, and a controller coupled to the optical sensor, the user interface, and the output device. In one or more embodiments, the controller includes one or more processors and a memory with instructions stored thereon, wherein the instructions, upon execution by the one or more processors, cause the one or more processors to perform one or more function. In one or more embodiments, the one or more instructions include receiving sensor input from the optical sensor. In one or more embodiments, the one or more instructions include determining the presence of a biological substance on the cleaning medium based on the sensor input, wherein the biological substance comprises feces. In one or more embodiments, the one or more instructions include generating a data signal based on the presence of the biological substance. In one or more embodiments, the one or more instructions include transmitting the data signal to the output device.

A method for testing a cleaning medium after wiping a perineum is also disclosed. In one or more embodiments, the method includes A method for testing a cleaning medium after wiping a perineum. In one or more embodiments, the method further includes presenting the cleaning medium to a camera. In one or more embodiments, the method further includes scanning the cleaning medium. In one or more embodiments, the method further includes processing an image of the cleaning medium. In one or more embodiments, the method further includes detecting a presence of feces on cleaning medium. In one or more embodiments, the method further includes communicating a status of the cleaning medium to the user

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims. In the drawings:

FIG. 1A is a drawing illustrating a cleaning medium scanner system, in accordance with one or more embodiments of the present disclosure;

FIG. 1B is a block diagram illustrating an organization of the system 100 and system componentry, in accordance with one or more embodiments of the disclosure;

FIG. 2 is a drawing illustrating a visual shield configured for use with the cleaning medium scanner system, in accordance with one or more embodiments of the present disclosure;

FIG. 3 is a drawing illustrating a front view of a toilet configured with the cleaning medium scanner system, in accordance with one or more embodiments of the present disclosure;

FIG. 4 is a drawing illustrating a front view of a toilet configured with the cleaning medium scanner system, with a user placing a cleaning medium near an optical sensor, in accordance with one or more embodiments of the present disclosure;

FIG. 5 is a drawing illustrating a close-up view of a cleaning medium scanner system in use with a shield, in accordance with one or more embodiments of the present disclosure;

FIG. 6 is a drawing illustrating a front view of a toilet with a detached optical sensor and display, in accordance with one or more embodiments of the present disclosure;

FIG. 7A is a drawing illustrating a plurality of result images of the display, in accordance with one or more embodiments of the present disclosure;

FIG. 7B is a drawing illustrating a control panel screenshot of the control panel, showing audio and display options, in accordance with one or more embodiments of the disclosure;

FIG. 8 is a drawing illustrating the system 100, including a stand that supports one or more components of the system; in accordance with one or more embodiments of the disclosure; and

FIG. 9 is a drawing illustrating a flowchart of a method for using the cleaning medium scanner system, In accordance with one or more embodiments of the present disclosure.

### DETAILED DESCRIPTION

Before explaining one or more embodiments of the disclosure in detail, it is to be understood that the embodiments are not limited in their application to the details of construction and the arrangement of the components or steps or methodologies set forth in the following description or illustrated in the drawings. In the following detailed description of embodiments, numerous specific details may be set forth in order to provide a more thorough understanding of the disclosure. However, it will be apparent to one of ordinary skill in the art having the benefit of the instant disclosure that the embodiments disclosed herein may be practiced without some of these specific details. In other instances, well-known features may not be described in detail to avoid unnecessarily complicating the instant disclosure.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only and should not be construed to limit the disclosure in any way unless expressly stated to the contrary.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of “a” or “an” may be employed to describe elements and components of embodiments disclosed herein. This is done merely for convenience and “a” and “an” are intended to include “one” or “at least one,” and the singular also includes the plural unless it is obvious that it is meant otherwise.

Finally, as used herein any reference to “one embodiment” or “some embodiments” means that a particular element, feature, structure, or characteristic described in connection with the embodiment is included in at least one embodiment disclosed herein. The appearances of the phrase “in some embodiments” in various places in the specification are not necessarily all referring to the same embodiment, and embodiments may include one or more of the features expressly described or inherently present herein, or any combination of sub-combination of two or more such features, along with any other features which may not necessarily be expressly described or inherently present in the instant disclosure.

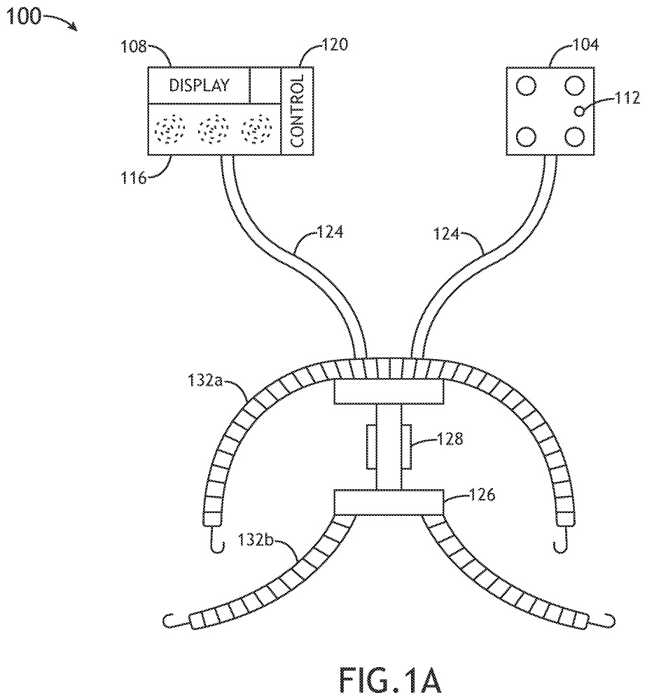

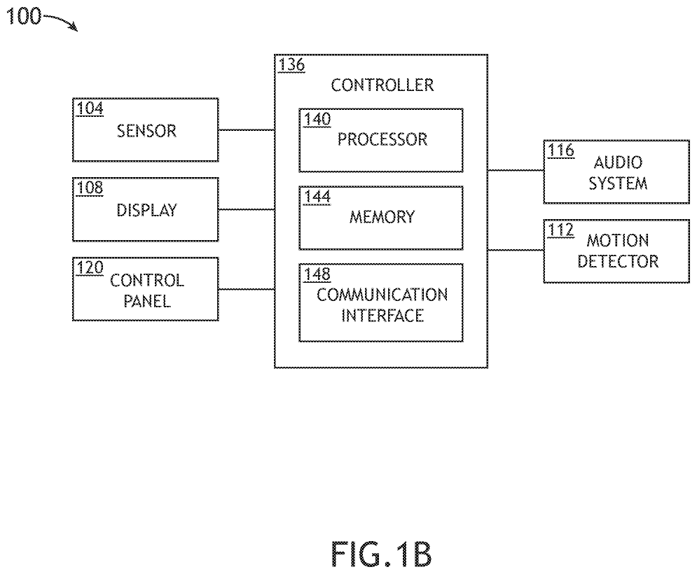

FIGS. 1-9 generally illustrate a system 100 for scanning a cleaning medium, in accordance with one or more embodiments of the present disclosure. The system 100 scans a cleaning medium (e.g., tissue paper, toilet tissue, wipes, or other cleaning products) after wiping the perineum, such as after defecation, and can determine whether the cleaning medium is soiled. The system 100 may also determine the type of biological substance (e.g., feces, urine, blood, cellular material, menstrual fluid, biomarkers, or other biological material) that has been picked up by the cleaning medium.

FIG. 1A illustrates the system 100, in accordance with one or more embodiments of the present disclosure. In embodiments, the system 100 includes an optical sensor 104 configured to scan a cleaning medium and transmit sensor input. For example, the optical sensor 104 may be configured to detect the condition of the tissue paper (e.g., soiled or unsoiled) and/or detect specific components of the waste matter on the tissue paper (e.g., feces, urine, or blood). The optical sensor 104 may be any type of sensor including but not limited to a colorimeter, a color sensor, a camera, or a laser scanner.

In some embodiments, the system 100 includes a display 108 configured to communicate with a user a message regarding the condition of the cleaning medium that was scanned by the optical sensor 104. For example, the display 108 may present a text message to the user that the cleaning medium is soiled. In another example, the display 108 may present an icon (e.g., a green circle) that the cleaning medium is clean. In another example, the display 108 may present a cartoon-like representation of the scanned cleaning medium.

In some embodiments, the system 100 includes a motion detector 112 configured. The motion detector 112 may be configured to detect an approach of a user of system 100, detect the presenting of the cleaning medium in front of the optical sensor 104, and/or a detect a toilet that is configured with the system 100.

In some embodiments, the system 100 includes an audio system 116 configured to communicate to the user the condition of the cleaning medium. For example, the audio system 116 may emit a beep, or a series or beeps, indicating the cleaning medium is clean. In another example, the audio system 116 may emit a buzzing noise that the cleaning medium is not clean. In another example, the audio system 116 may emit a voice message describing the condition of the cleaning medium. Any type of audio communication configured to communicate the condition of the cleaning medium to the user is possible. The audio system 116 and the display 108 may be independently or collectively termed an output device.

In some embodiments, the output device is configured as a mobile device. For example, the system 100 may be configured to communicate wirelessly with a smartphone, tablet, or other electronic device of a user.

In some embodiments, the system 100 includes a control panel 120 (e.g., a user interface). The control panel 120 may be configured as a user interface device. For example, the user may control aspects of the display 108, the motion detector 112, and/or the audio system 116 via the control panel 120. The control panel 120 may use touch-sensitive technology, such as for touch displays, or physically interactive controls.

The system 100 may be configured as having a singular housing containing one or more components (e.g., the optical sensor 104, the display 108, the audio system 116, or the control panel 120). However, the system may also be configured with multiple housings for one or more components. For example, the optical sensor 104 may be disposed in a housing separate than the control panel 120 and communicatively linked via a wireline connection, (disclosed within conduit 124) or a wireless connection. In another example, one or more components may have their own respective housing, with the one or components interconnecting to each other via adjustable rods. Power to the system 100 may be supplied via battery (e.g., via battery pack 126) or an external power source (e.g., 110V/120V wall outlet) connected via the conduit 124 or other power cable. In some embodiments, one or more, or all, components of the system 100 are incorporated into a toilet or bidet. Therefore, the description herein should not be interpreted as a limitation of the present disclosure, but as an illustration.

The display 108 may be formed of any type of display including but not limited to a liquid crystal display (LCD) (e.g., a light-emitting diode (LED) backlit LCD, a thin-film resistor (TFT) LCD, or a quantum dot LED (QLED) display), an LED display (e.g., organic light-emitting diode (OLED) display, or active-matrix OLED (AMOLED) display), a cathode ray tube display, a digital light processing (DLP) display, a plasma display panel (PDP), or a microLED display. The display 108 may be configured as any size or shape.

In some embodiments, the system 100 includes a main support 128 with straps 132a-b configured to secure and/or anchor the system 100 to the toilet. The main support 128 and straps 132a-b may be configured as any type of attachment apparatus and may include any type of attachment technology including but not limited to hooks, snaps, buckles, and/or hook-and-loop (e.g., Velcro).

FIG. 1B is a block diagram illustrating an organization of the system 100 and system componentry, in accordance with one or more embodiments of the disclosure. In some embodiments, the system 100 includes a controller 136 configured to provide processing functionality for the system 100. The controller 136 may be communicatively linked to any of the components of the system 100 (e.g., the optical sensor 104, the display 108, the control panel 120, the audio system 116, and/or the motion detector 112).

The controller 136 may include one or more processors 140, memory 144, and a communication interface 148. The controller 136 may communicate with one or more components of the system 100 via wireline or wireless methods. The one or more processors 140 may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors 140 may include any device configured to execute algorithms and/or instructions (e.g., program instructions stored in memory 144). In one embodiment, the one or more processors 140 may be embodied as a desktop computer, mainframe computer system, workstation, image computer, parallel processor, networked computer, or any other computer system configured to execute a program configured to operate or operate in conjunction with the system 100, as described throughout the present disclosure. Moreover, different subsystems of the system 100 may include a processor 140 or logic elements suitable for carrying out at least a portion of the steps described in the present disclosure. Therefore, the above description should not be interpreted as a limitation on the embodiments of the present disclosure but merely as an illustration.

The memory 144 can be an example of tangible, computer-readable storage medium that provides storage functionality to store various data and/or program code associated with operation of the controller 136 and/or other components of the system 100, such as software programs and/or code segments, or other data to instruct the controller and/or other components to perform the functionality described herein. Thus, the memory 144 can store data, such as a program of instructions for operating the system 100 or other components (e.g., instructions that, upon execution by the one or more processors 140, cause the one or more processors 140 to perform one or more functions of the system 100. It should be noted that while a single memory 144 is described, a wide variety of types and combinations of memory 144 (e.g., tangible, non-transitory memory) can be employed. The memory 144 can be integral with the controller 136, can comprise stand-alone memory, or can be a combination of both. Some examples of the memory 144 can include removable and non-removable memory components, such as a programmable logic device, random-access memory (RAM), read-only memory (ROM), flash memory (e.g., a secure digital (SD) memory card, a mini-SD memory card, and/or a micro-SD memory card), solid-state drive (SSD) memory, magnetic memory, optical memory, universal serial bus (USB) memory devices, hard disk memory, external memory, and so forth.

The communication interface 148 can be operatively configured to communicate with components of the controller 136 and other components of the system 100. For example, the communication interface 148 can be configured to retrieve data from the controller 136 or other components, transmit data for storage in the memory 144, retrieve data from storage in the memory 144, and so forth. The communication interface 148 can also be communicatively coupled with controller 136 and/or system elements to facilitate data transfer between system components.

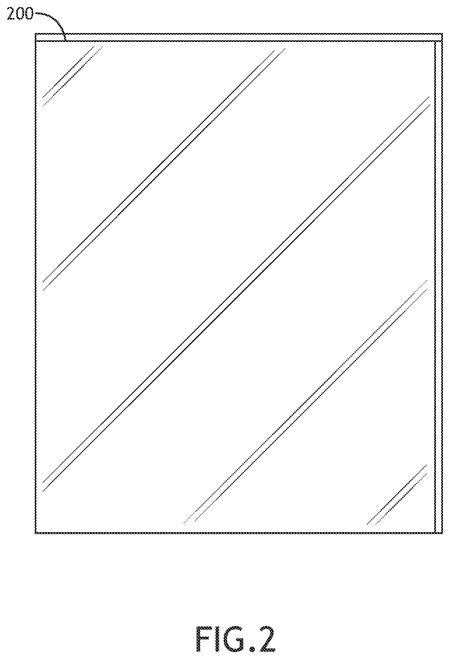

In some embodiments the system 100 includes a shield 200 configured to block the view of the cleaning medium being scanned, as shown in FIG. 2. om accordance with one or more embodiments of the disclosure. For example, a person who dislikes or is sickened by the viewing fecal may use the shield 200 to avoid seeing the soiled cleaning medium (e.g., soiled tissue paper).

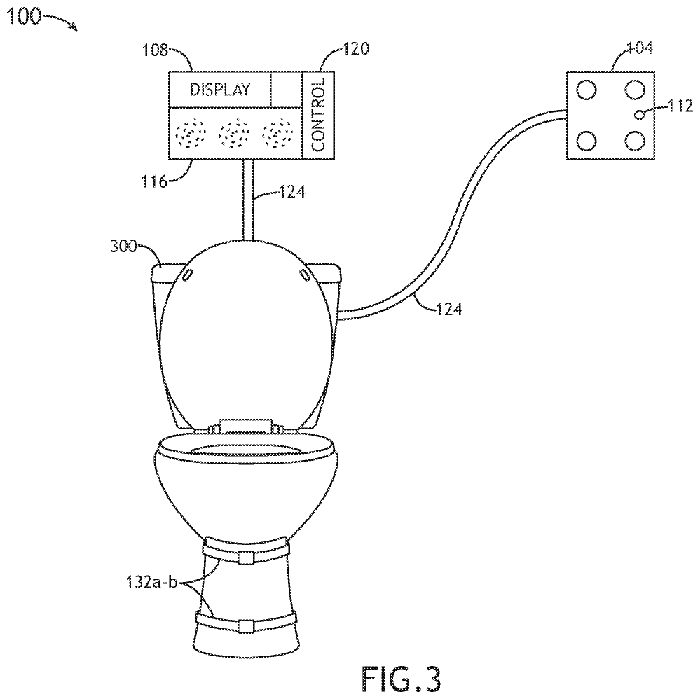

FIG. 3 illustrates a front view of a toilet 300 configured with the system 100, in accordance with one or more embodiments of the present disclosure. In this embodiment, the display 10, audio system 116, and the control panel 120 are encased in a main housing above the toilet 300, while the optical sensor 104 and motion detector 112 are housed within a separate paper sensor housing that is physically connected to the main housing via one or more adjustable rods. However, multiple combinations/arrangements of system 100 components are possible. Therefore, the description herein should not be interpreted as a limitation of the present disclosure, but as an illustration.

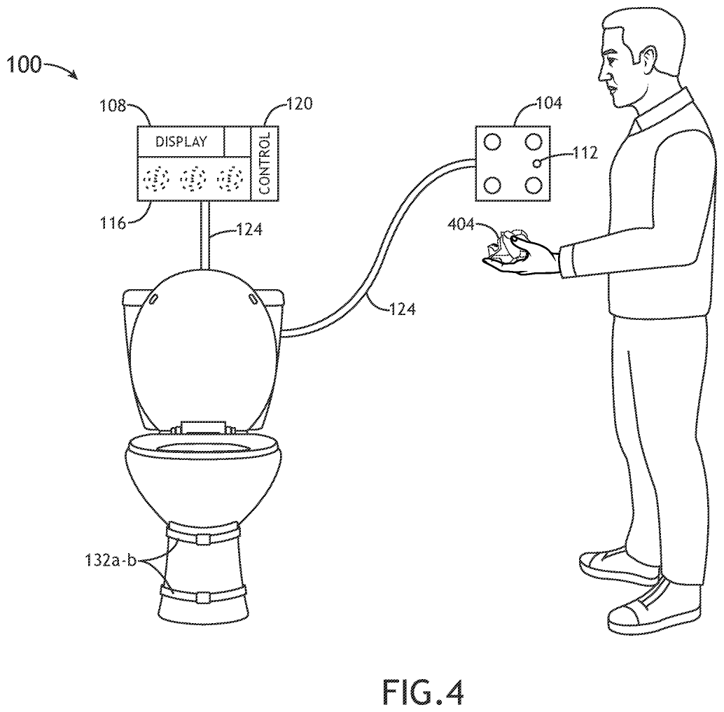

FIG. 4 illustrates a front view of the toilet configured with the system 100, with a user 400 placing the cleaning medium 404 (e.g., toilet paper) near the optical sensor 104, in accordance with one or more embodiments of the present disclosure. For example, a user 400 may hold a balled-up mass of cleaning medium 404 in front of the optical sensor 104. The optical sensor 104 then scans the cleaning medium 404 and the result of the scan is shown on the display 108.

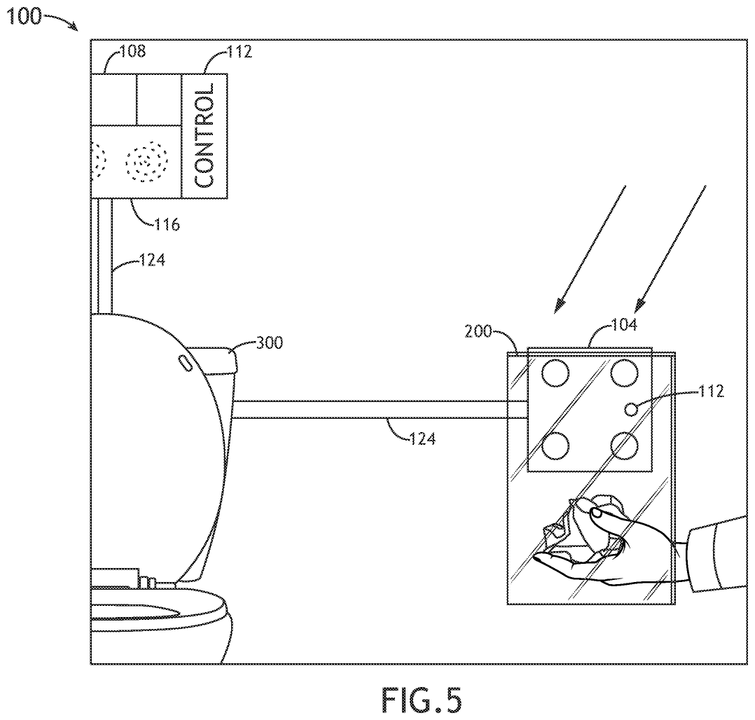

FIG. 5 illustrates a close-up view of the tissue paper sensor in use with the shield 200, in accordance with one or more embodiments of the present disclosure. The shield obstructs the user 400 from viewing the cleaning medium 404. The shield 200 may be of any size or shape, and may be coupled to the system 100 by any means (e.g., a hinge, or a bracket)

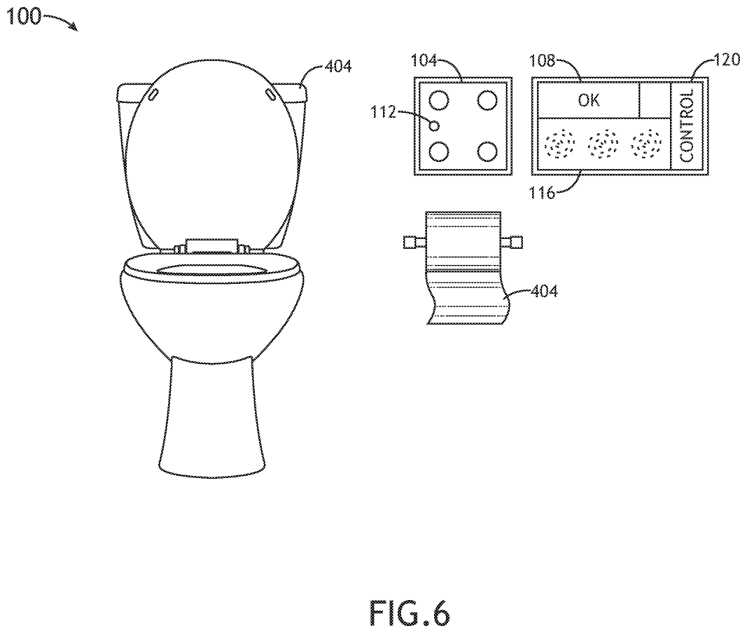

FIG. 6 are drawings illustrating a front view of a toilet with a detached tissue paper sensor and display, in accordance with one or more embodiments of the present disclosure. In some embodiments, the paper sensor 104 and the main housing that includes the other components of the system 100 (e.g., the display 108, the motion detector 112, audio system, 4, and the control panel 120) are physically separated from the other but are communicatively coupled via a wireless connection.

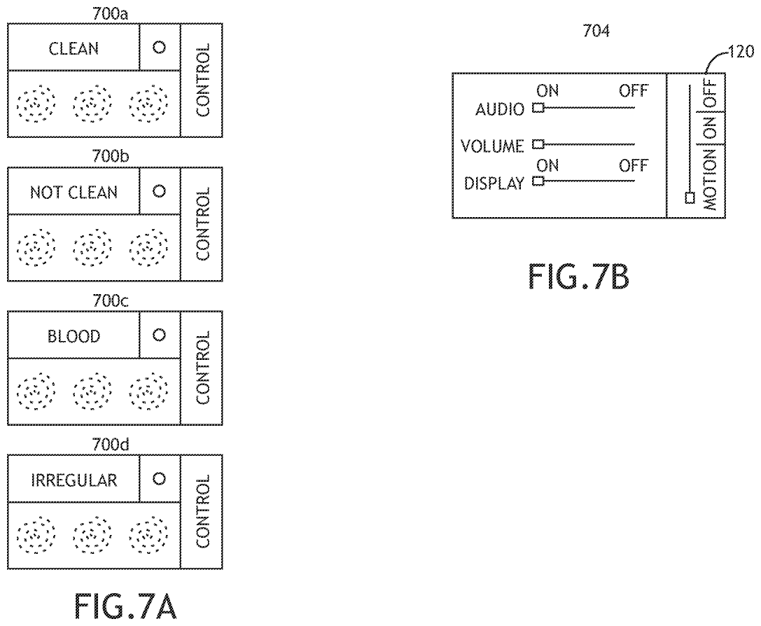

FIG. 7A are drawings illustrating a plurality of result images of the display, in accordance with one or more embodiments of the present disclosure. The display 108 may show a number of screenshots (700a-d) based on the outcome of the scan by the optical sensor 104. For example, the display may display a “clean” screenshot 700a indicating that the paper is clean. In another example, the display may display a “not clean” screenshot 700b indicating that the paper is soiled. In another example, the display may display a “Blood” warning (e.g., screenshot 700c) indicating the presence of blood on the paper. In another example, the display may display an “irregular” warning 700d, indicating that irregular cells or other debris is on the paper. For instance, the “irregular” warning 700d may indicate the presence of cancer cells.

The system 100 may be configured to determine the results of the scan (e.g., clean, not clean, blood, irregular) using any technology. For example, the system 100 may be use colorimetric data received from the sensor 14 to determine whether the substance on the cleaning medium 404 contains blood or feces (e.g., determine the presence of blood or feces on the cleaning medium 404 based on input from the optical sensor.). In another example, the optical sensor 104 may include an illumination source that illuminates the cleaning medium 404 at one or more wavelengths. The illumination may serve to increase the quality of image taken by the optical sensor 104 and may be used to identify specific components of material on the cleaning medium, such as vaginal fluids, which are known to fluoresce.

The system 100 may also be configured to determine specific characteristics of the biological material, such as a color, an area, an amount, or a concentration. For example, the system 100 may be configured to determine the area upon the cleaning medium 404 that is coated with the biological material. The system 100 may also be configured to store data from the scans/images within a database (e.g., utilizing the memory 144 of the system 100 or an external memory). In this manner, the system 100 may then be able to compare historical scan data and alert the user of a change in one of more characteristics of the biological material. For example, the system may determine that the cleaning medium 404 is coated with a higher percentage, concentration, or amount of blood that the last time the system 100 was used, and would alert the user 400 accordingly.

FIG. 7B is a drawing illustrating a control panel screenshot 704 of the control panel 120, showing audio and display options, in accordance with one or more embodiments of the disclosure. For example, the control panel 120 may be configured with an option to turn on/off, or adjust, the audio output of the audio system 116. In another example, the control panel 120 may be configured to turn on/off, or adjust the display 108. In another example, the control panel 120 may configured to turn off/on the motion detector 112.

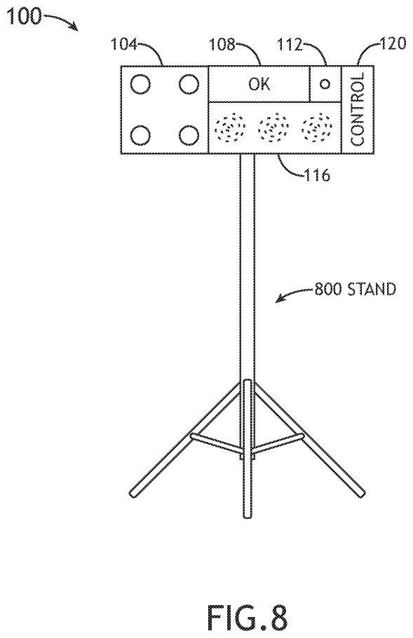

FIG. 8 is a drawing illustrating the system 100, including a stand 800 that supports one or more components of the system 100 (e.g., the optical sensor 104, the display 108, the audio system 116, the motion detector 112, and/or the control panel 120). The stand 800 may be of any size, shape, or type. For example, the stand 800 may be configured as a metal tripod.

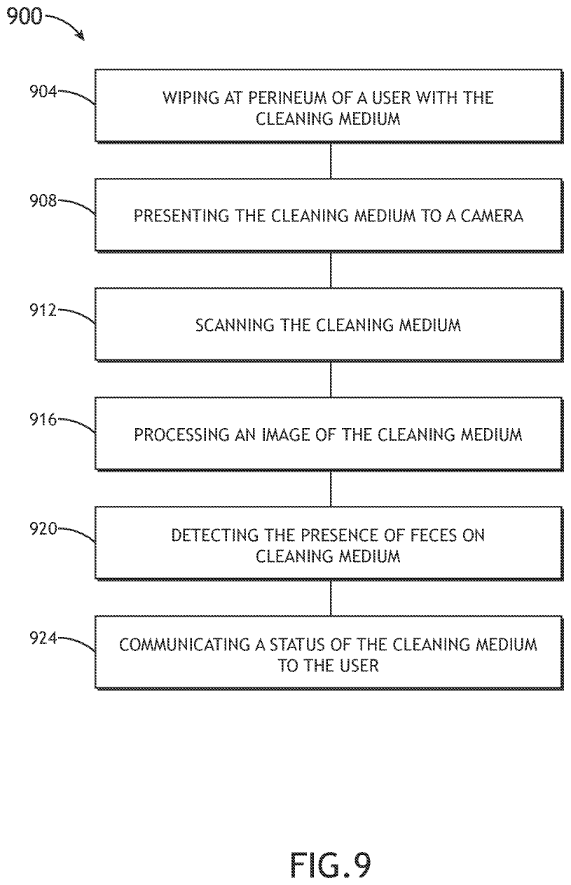

FIG. 9 illustrates a flowchart of a method 900 method for testing a cleaning medium after wiping a perineum, In accordance with one or more embodiments of the present disclosure. In embodiments, the method includes a step 904 of wiping the anus with a cleaning medium 404. The cleaning medium 404 may include any type of cleaning material, such as toilet paper.

In embodiments, the method 900 further includes a step 908 of moving the cleaning medium 404 in front of the optical sensor 104. The optical sensor 104 may automatically scan the cleaning medium (e.g., the system automatically activated via the motion detector 112). The user 400 may also activate the optical sensor 104 via the control panel 120.

In embodiments, the method 900 further includes a step 912 of scanning the cleaning medium and a step 916 of processing an image of the cleaning image. The scanning of the medium may be performed by the scanner, while the processing of the image of the cleaning medium 404 may be performed by either the scanner, or by one or more processors 140.

In embodiments, the method 900 further includes a step of 916 detecting the presence of feces on the cleaning medium 404. For example, the system 100 include image analysis software to determine the presence of fecal material on the cleaning medium. The software may be configured to adjust to different types or colors of cleaning mediums, as different types of toilet paper often have different shades of white. The system 100 may then generate a data signal based on the presence of the biological substance and transmit the data signal to the output devices, such as the display 108.

In embodiments, the method 900 further includes a step 920 of communicating a status of the cleaning medium 404 to the user 400. For example, the system 100 may communicate to the user 400 that the presence of feces on the cleaning medium 404 (e.g., soiled on unsoiled). The system 100 may also communicate to the user the presence of blood, urine, menstrual fluid, cellular material, or a biomarker on the cleaning medium 404. The system 100 may also communicate to the user the presence of an irregular or abnormal result, in which case the system 100 may either automatically consult medical personnel about the result, or instructing the user 400 to consult the medical personnel.

the display 108 may display a message of the results of the scan. In embodiments, the method further includes informing the user to consult a doctor based on the results of the scan.

It is to be understood that embodiments of the methods disclosed herein may include one or more of the steps described herein. Further, such steps may be carried out in any desired order and two or more of the steps may be carried out simultaneously with one another. Two or more of the steps disclosed herein may be combined in a single step, and in some embodiments, one or more of the steps may be carried out as two or more sub-steps. Further, other steps or sub-steps may be carried in addition to, or as substitutes to one or more of the steps disclosed herein.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

Although particular embodiments of this invention have been illustrated, it is apparent that various modifications and embodiments of the invention may be made by those skilled in the art without departing from the scope and spirit of the foregoing disclosure. It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Accordingly, the scope of the invention should be limited only by the claims appended hereto.

What is claimed is:

1. A system comprising:
an optical sensor configured to scan a cleaning medium and transmit a sensor input;
an output device;
a user interface; and
a controller coupled to the optical sensor, the user interface, and the output device, comprising:
one or more processors; and
a memory with instructions stored thereon, wherein the instructions, upon execution by the one or more processors, cause the one or more processors to:
receive the sensor input from the optical sensor;
determine a presence of a biological substance on the cleaning medium based on the sensor input, wherein the biological substance comprises feces;
generate a data signal based on the presence of the biological substance; and
transmit the data signal to the output device.

2. The system of claim 1, wherein the biological substance further comprises blood.

3. The system of claim 1, wherein the biological substance further comprises cellular material.

4. The system of claim 1, wherein the biological substance further comprises a biomarker.

5. The system of claim 1, wherein the system is configured to determine a characteristic of the biological substance comprising at least one of an area, a color, an amount, or a concentration.

6. The system of claim 1, wherein the output device is configured as a display.

7. The system of claim 1, wherein the output device is configured as an audio system.

8. The system of claim 1, further comprising a main support configured to anchor a system component to a toilet.

9. The system of claim 1, further comprising a stand configured to support a system component.

10. The system of claim 1, further comprising a shield configured to visually shield a view of the cleaning medium from a user.

11. The system of claim 1, wherein the output device is configured to output a message to a user that the cleaning medium if soiled or clean.

12. The system of claim 1, wherein the output device is configured as a mobile device.

13. The system of claim 1, wherein the optical sensor is configured as a camera.

14. The system of claim 1, further comprising a database configured to store the data signal.

15. The system of claim 1, wherein the cleaning medium is configured as tissue paper.

16. A method for testing a cleaning medium after wiping a perineum of a user comprising:
wiping the perineum of the user with the cleaning medium;
presenting the cleaning medium to a camera;
scanning the cleaning medium;
processing an image of the cleaning medium;
detecting a presence of feces on cleaning medium; and
communicating a status of the cleaning medium to the user.

17. The method of claim 16, further comprising detecting a presence of blood, cellular material, or a biomarker.

18. The method of claim 16, further comprising detecting an abnormal result.

19. The method of claim 18, further comprising at least of consulting medical personnel, or instructing the user to consult the medical personnel, of the abnormal result.

20. The method of claim 16, wherein the status of the cleaning medium comprises soiled and unsoiled.
